(19) United States
(12) Patent Application Publication
(10) Pub. No.: US 2005/0027176 A1
(43) Pub. Date: Feb. 3, 2005

(54) Optical in vivo analyte probe using embedded intradermal particles

(75) Inventor: Jinchun Xie, (US)

Correspondence Address: Michael D. Van Loy;Skymoon Research and Development, LLC, Intellectual Property Department, 3045 Park Boulevard, Palo Alto, CA, 94306, US

(73) Assignee: Skymoon Research & Development, LLC

(21) Appl. No.: 10/909,571

(22) Filed: Aug. 2, 2004

Related U.S. Application Data
(60) Provisional application No. 60/491,356, filed on Jul. 31, 2003.

(51) Int. Cl. A61B 5/00

(52) U.S. Cl. 600/316; 600/310

(57) ABSTRACT

A system and method of in vivo detection and quantification of one or more analytes. Small particles comprising a surface-active monolayer coating are embedded in the dermis. The surface-active monolayer acts to pre-concentrate the analyte by adsorbing the analyte from bulk solution. The concentrated analyte is more readily detected and quantified by one or more spectroscopic methods such as Raman spectroscopy

### RELATED APPLICATIONS

This application claims the benefit of U.S. Provisional Patent Application Ser. No. 60/491,356, filed on Jul. 31, 2003, the disclosure of which is hereby incorporated by reference in its entirety.

### FIELD OF THE INVENTION

The present invention relates generally to the field of in vivo detection and/or quantification of analytes. More specifically, the present invention relates to systems and methods for detecting, analyzing, and/or quantifying concentrations of compounds by measuring the spectral responses of sensitized particles embedded within tissue such as the skin.

### BACKGROUND OF THE INVENTION

Raman spectroscopy is a powerful tool for revealing specific molecular signatures from a complex system. For glucose in vivo detection, a Raman excitation laser emitting in the near IR region of the spectrum can penetrate into tissues to probe the molecular vibration which can not be done by IR spectroscopy due to strong water absorption. However, Raman scattering is a nonlinear process and tends to have a very small cross section (i.e., approximately 10⁻³⁰ cm⁻² sr⁻¹ molecule⁻¹). Two known approaches for enhancing Raman scattering processes are surface enhanced Raman spectroscopy (SERS) and UV resonance enhanced Raman. UV resonance enhanced Raman is less appropriate for in vivo detection applications because of limitations in the tissue transparency window. Specifically, UV radiation is strongly absorbed by human tissue, and glucose does not undergo electronic resonance in the visible or near-infrared regions of the spectrum. SERS analysis of glucose using silver nano-particles may enhance Raman scattering by more than million times.

The historic difficulty of SERS detection of glucose in vivo using prior art methods may be attributable to the weak or nonexistent binding of glucose to bare silver surfaces. The normal Raman cross section of glucose should provide a sufficient signal for detection and quantification. Experiments performed by Weaver and co-workers (Mrozek, M. F.; Weaver, M. J. Anal. Chem. 2002, 74, 4069-4075) indicate that glucose must be trapped in a junction between the roughened electrode and the colloidal nano-particles. The stability of SERS activity has been unambiguously demonstrated for bare silver films over nano-sphere (AgFON) surfaces over a potential range from Ag oxidation to H₂ evolution and at high temperatures in ultrahigh vacuum, as reported in Dick, L. A.; McFarland, A. D.; Haynes, C. L.; Van Duyne, R. P. J. Phys. Chem. B 2002, 106, 853-860, and Litorja, M.; Haynes, C. L.; Haes, A. J.; Jensen, T. R.; Van Duyne, R. P. J. Phys. Chem. B 2001, 105, 6907-6915.

Because of the advantages of SERS detection techniques for glucose, a system and method for applying this detection technique in vivo is desirable. The present invention provides a novel approach to the problem, using SERS-active nano-particles that are implanted in a subject in a manner similar to tattooing.

### SUMMARY OF THE INVENTION

The present invention provides for in vivo optical detection of analytes (e.g., glucose) by embedding small particles within the dermis of the skin. The small particles are made sensitive to the analyte of interest by construction. For example, silver-coated nanoparticles further covered with a self-assembled molecular monolayer are suitably sensitized for the detection of glucose. The particles embedded within the dermis are preferably probed by surface enhanced Raman spectroscopy, although other optical probe techniques can be employed.

A first embodiment of the present invention provides a method for in vivo detection of an analyte in a subject. Sensitized particles having an adsorptive affinity for the analyte are embedded within the skin of the subject. The analyte concentration at the surface of the sensitized particles is greater than the analyte concentration in the subject. The sensitized particles are optically illuminated with excitation radiation. Optical radiation emitted by the sensitized particles is collected and measured. The emitted radiation is analyzed to detect the analyte and/or quantify the concentration of the analyte in the subject.

In an alternative embodiment, an apparatus is provided for in vivo determination of an analyte. The apparatus comprises a housing that is attachable to a subject's arm by a strap such that a face of the housing is approximately flush with a skin surface on the subject's arm. The housing contains an optical path for conveying an incident beam of excitation light from an excitation source and for conveying collected scattered radiation to a detector. This optical path comprises an angled mirror to direct the incident beam approximately perpendicular to the skin and a focusing lens that focuses the incident beam on a tattoo spot in the skin and focuses the scattered radiation emitted from the tattoo spot

### BRIEF DESCRIPTION OF THE DRAWINGS

Other objects and advantages of the present invention will become apparent upon reading the detailed description of the invention and the appended claims provided below, and upon reference to the drawings, in which:

FIG.1a is a schematic diagram showing placement of a monitoring device according to the present invention designed to be worn on the wrist.

FIG.1B is a schematic diagram showing placement of a monitoring device according to the present invention designed to be worn on the upper arm.

FIG.2 is a schematic diagram showing silver nano particles made by coating silver over uniform polystyrene latex nano-spheres that are further covered with a self-assembled molecular monolayer.

FIG.3 is a schematic diagram showing a view of human skin structure and physiology.

FIG.4 is a schematic diagram showing the distribution of tattoo ink particles at various stages of a tattoo implanting process for use with one or more of the embodiments of the present invention.

FIG.5 is a schematic diagram showing a side view of an embodiment of the invention in which excitation light from a laser source passes through a collimating lens, beam splitter, right angle mirror and a focusing lens onto a tattoo spot.

FIG.6 is a chart showing calculated contour lines of absorbed energy based on a skin model, for the case of a narrow incident beam (the 1/^{e2} radius of the incident beam is approxaimtely 0.01 cm).

FIG.7 is a schematic diagram showing a modification of the embodiment shown in FIG.6, wherein a window plate is pressed against the skin.

FIG.8 is a schematic diagram showing a top view (i.e., looking down on the skin) of the apparatus shown in FIG.6.

FIG.9 is a chart showing Raman spectra of various compositions.

FIG.10 is a schematic diagram showing a modification of the embodiment of FIG.8, wherein multiple spectral bands of the Raman radiation emitted by particles within the dermis are measured.

FIG.11 is a chart showing Raman spectra of various components of the skin, along with a SERS glucose spectrum for comparison.

### DETAILED DESCRIPTION OF PREFERRED EMBODIMENTS

1. Selective Enhanced Glucose Detection

Detection of glucose (or other analytes) using SERS presents two potential problems. Silver nano-particles (or other sensitized small particles) must be implanted in the human body to effectively make contact with glucose, and the nano-particles must be sensitized to glucose, which generally does not readily adhere to silver surfaces. The present invention solves the first problem by applying well-known tattooing techniques for implantation of sensitized particles.

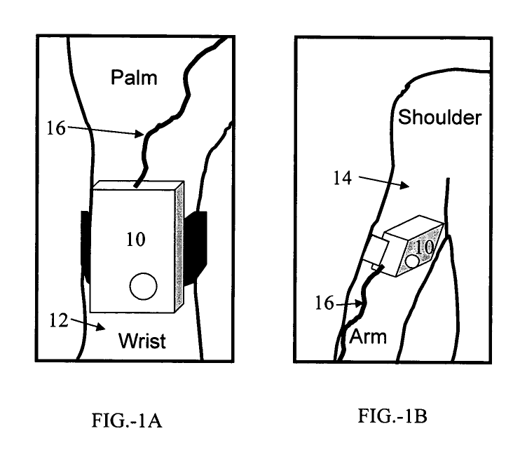

Referring to FIG.1A and FIG.1B, a monitor 10 according to the present invention may advantageously be worn on the inner wrist 12 as shown in FIG.1A, on the inner upper arm 14 as shown in FIG.1B, or, alternatively, on some other portion of the body where close and consistent skin contact may be maintained. For the examples shown in FIG.1A and FIG.1B, contact is made to skin on the inner part of the wrist or arm, respectively. A fiber cable 16 connects the monitor unit to a laser source (not shown) that provides incident radiation and to a spectrograph analyzer (also not shown) for receiving and processing the optical spectrum.

According to one embodiment of the present invention, nano-particles may be implanted within the dermis of the skin as tattoo ink for selective glucose detection via SERS. A round tattoo spot may be made on the inner arm where the skin is thin and lightly colored. The spot is advantageously of approximately 1 mm diameter. The glucose Raman reader may advantageously be configured as a watch on the lower arm (i.e., near wrist as shown on FIG.1A) or as a device strapped to the upper arm (i.e., above elbow as shown on FIG.1B). This upper arm device may resemble a personal portable audio cassette player in shape and/or size. The optical detector may be configured similarly to a compact disk reading head. By moving the detector, the tattoo mark may advantageously be aligned to optimize detection. The tattoo spot may be viewed through a magnified viewport.

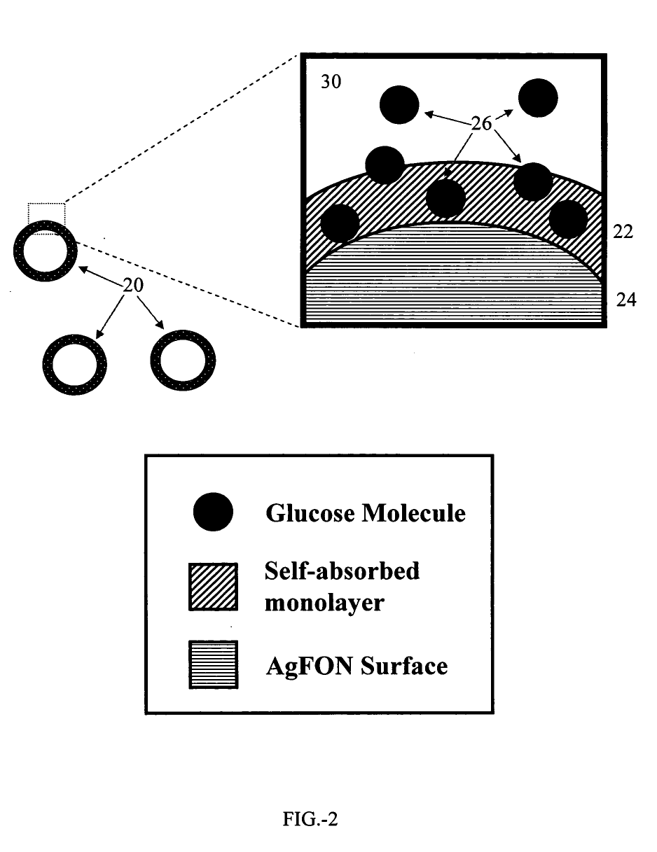

The second issue with in vivo SERS quantification of glucose involves sensitization of the implanted nano-particles. To increase glucose interaction with a “silver film over nanosphere” (AgFON) surface, a self-assembled monolayer (SAM), as taught by Shafer-Peltier et al, (J. Am. Chem. Soc. 2003, 125 588-593), the disclosure of which is incorporated herein by reference in its entirety, may be formed on an AgFON surface to preconcentrate the analyte of interest. An example of this process is shown schematically in FIG.2, which is reproduced from the Shafer-Peltier paper. Referring to FIG.2, the sensitized nano-particles 20 comprisea SMA layer 22 over an AgFON surface 24. In vivo, glucose (or other analyte) molecules 26 adsord into and onto the Sam layer 22 from the bulk solution 30. Such preconcentration may be thought of as analogous to that used to create the stationary phase in high performance liquid chromatography (HPLC). Implementing such a partition layer has three advantages: (1) the SAM stabilizes the Ag surface against oxidation; (2) the SAM is exceedingly stable; and (3) preconcentration functionality is built in and tailorable by synthetic control of the partition layer.

Surfactant-free white carboxyl-substituted polystyrene latex nanospheres with diameters of 400 nm were coated with silver metal films in the aforementioned Shane-Peltier study. The mass thickness of Ag in all cases was approximately 200 nm. Fresh AgFON samples were incubated in approximately 1 mM solutions of the partition layer self assembled monolayers (SAMs) in ethanol for greater than approximately 12 h for complete coverage. The authors report the growth of a layer of “hair-like” molecular chains on the silver nano particle surface to make up a self-assembled monolayer (SAM). The length of the SAM chains is about 4 nm, comparable to the spatial extent of the surface plasmons, which are exploited in surface enhanced Raman spectroscopy. Various SAM layers were tried, but only straight chain alkanethiols were found to be effective in enhancing the sensitivity to glucose. The SAM selectively adsorbs glucose within its hair-like structure leading to pre-concentration, namely the concentration of glucose in the SAM layer is substantially greater than that in bulk solution. This pre-concentration effect is approximately linearly proportional to the concentration of glucose in the solution. Kinetic analysis also shows a relatively fast (approximately 10 msec) process to reach equilibrium. Therefore, a SAM layer selectively enhances the glucose Raman signal. The self-assembled monolayers (SAMs) used in this work are known to be extremely stable by themselves and as adsorbates on AgFON surfaces. While glucose analysis is described in the instant application to illustrate both the operation and potential advantages of the present invention, other analytes may be detected and quantified in vivo based on the teachings provided herein. One of skill in the art would be able to select and appropriate SAM/sensitized nano-particle system for use with additional analytes using routine experimentation.

A possible kinetic scheme for a SERS-based glucose sensor according to the present invention is

$${G}_{bulk} + S \frac{{k}_{ads}}{\overline{{k}_{des}}} {G}_{ads} \frac{{k}_{p}}{\overline{{k}_{- p}}} {G}_{part} \qquad ( 1 )$$

In general, glucose must diffuse from bulk solution, G_{bulk}, to the solution/alkanethiol SAM interface, S, where it is adsorbed, G_{ads}, and then partitioned, Gₚₐᵣₜ, into the SAM. IN equation 1, k_{ads} is the bimolecular rate constant for adsorption of glucose at the solution/SAM interface, k_{des} is the unimolecular rate constant for desorption of glucose from the solution/SAM interface, kₚ is the rate constant for partitioning of glucose into the SAM, and k₋ₚ is the rate constant for departitioning of glucose from the SAM. Assuming that the diffusion step is rate limiting, a glucose detector according to the present invention may have a response time of approximately 10 millisecond time.

2. Human Skin Tissue Composition, Structure and Optical Properties

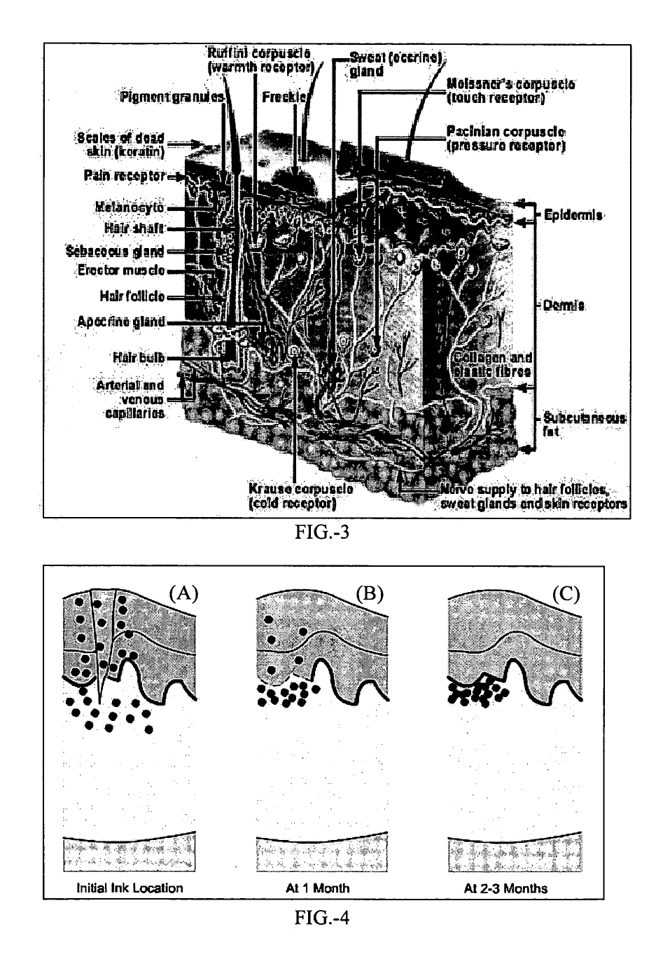

The structure and properties of skin vary considerably in different parts of the body. A typical structure is shown schematically in FIG.3. The skin is divided into three layers: the epidermis, dermis, and subcutaneous fat, each layer having its own sublayers. The outermost layer of the epidermis is composed of a relatively thin, but rough, protective top layer of dead and dry skin cells, known as the stratum corneum or horny layer. The remainder of the epidermis, including the stratum lucidum, stratum granulosum and stratum spinosum, is made up of cells called keratinocytes as well as melanocytes, which are pigment cells responsible for skin pigmentation. The thickness of the epidermis varies from approximately 0.1 mm in the eyelids to nearly approximately 1 mm on the palms and soles (Goldsmith L A, Physiology, Biochemistry and Molecular Biology of the Skin, 2^{nd} edition, Oxford University Press, 1991). The dermis comprises a variety of cells, fibers, amorphous ground substance, nerves, oil glands, sweat glands, blood capillaries and vessels, hair roots, and other bodily fluids. The upper layer of the dermis, known as the papillary dermis, contains the vascular network and sensory nerve endings, whereas the deeper layer, known as the reticular dermis, consists mainly of a loose connective structure and epithelial-derived structures such as glands and follicles. The thickness of the dermis varies from approximately 0.3 mm in the eyelids to approximately 3 mm in the palm and soles. Subcutaneous fat is composed of fat cells, which form a cushioning layer between the skin and the deeper muscles. The subcutaneous fat layer also contains abundant blood flow.

The papillary dermis is an advantageous location for glucose detection because it contains the vascular network. The papillary dermis is also the layer in which tattoo ink particles tend to remain, making tattooing a suitable method for implanting sensitized small particles into the human body. By implanting nano-silver particles as tattoo ink, the particles will have sufficient contact with fresh human fluid containing a representative concentration of glucose. As a result, tattoo ink of nano-silver particles will exchange glucose concentration with extracellular fluid, which is next to the capillary vascular network.

3. “Tattoo” Injection of SERS-Enhanced Detection Particles

Tattoo formation. Tattoos are an ancient art form with origins that trace back as far as the Stone Age (12,000 BC). Tattoos have remained popular throughout time and across many cultures and continents. FIG.4 schematically illustrates a generic tattoo forming process. This description, representative of tattooing procedures in general, is published at www.bmezine.com/news/edit/A30205/arttatto.html. Tattoo particles are initially scattered around the pinch wound and are eventually grouped under the epidermis within the upper dermis (papillary layer). When ink is first deposited, as shown in panel (A) of FIG.4, ink is found in all layers of the epidermis, as well as in the top layer of the dermis. As time progresses after the initial ink deposit, the ink particles are removed from the epidermis, but they generally remain the dermis, as shown in panes (B) and (C) of FIG.4. Within the dermis, a prominent network of connective tissue surrounds each of the fibroblasts that contain ink particles, effectively entrapping and immobilizing the cells. The life span of these fibroblasts varies and may persist throughout the individual's life.

Tattoo ink. Tattoo ink is remarkably non-reactive histologically, despite widespread use by tattoo artists of a variety of pigments of unknown purity and identity. The most used tattoo inks are nano-particles of inorganic compounds, such as minerals. Tattoo pigment granules are typically composed of particles ranging from approximately 2 to 400 nm in diameter. The most common particle size is approximately 40 nm, less common are approximately 2 to 4 nm particles (slightly more electron dense), and least common are 400-nm particles, which are very electron dense with a crystalline structure. A study of freshly implanted eyeliner tattoo ink revealed particles in the extracellular matrix with diameters of approximately 0.1 to 1.0 μm, although the average particle size in the pigment vial prior to implantation was approximately 0.25 μm.

TABLE I
Some tattoo inks with color and chemical contains as well as their
source (from above referenced www.bmezine.com document).

| Color | Substance | Comment |
|---|---|---|
| Black | Iron Oxide (Fe₃O₄) | Natural black pigment is made from |
| | Iron Oxide (FeO) | magnetite crystals, powdered jet, wustite, |
| | Carbon | bone black, and amorphous carbon from |
| | Logwood | combustion (soot). Black pigment is |
| | | commonly made into India ink. Logwood |
| | | is a heartwood extract from Haematoxylon |
| | | campechisnum, found in Central |
| | | America and the West Indies. |
| Brown | Ochre | Ochre is composed of iron (ferric) oxides |
| | | mixed with clay. Raw ochre is yellowish. |
| | | When dehydrated through heating, ochre |
| | | changes to a reddish color. |
| Red | Cinnabar (HgS) | Iron oxide is also known as common rust. |
| | Cadmium Red | Cinnabar and cadmium pigments are high- |
| | (CdSe) | ly toxic. Naphthol reds are synthesized |
| | Iron Oxide (Fe₂O₃) | from Naphtha. Fewer reactions have been |
| | Napthol-AS | reported with naphthol red than the other |
| | pigment | pigments, but all reds carry risks of |
| | | allergic or other reactions |
| Orange | disazodiarylide | The organics are formed from the con- |
| | and/or | densation of 2 monoazo pigment mole- |
| | disazopyrazolone | cules. They are large molecules with good |
| | cadmium seleno- | thermal stability and colorfastness. |
| | sulfide | |

Silver nano-particles may be prepared with sizes very similar to those of commonly used tattoo ink particles. Chemically, silver nano-particles, advantageously coated with a suitable SMA layer, may be made non-reactive in human tissue like other tattoo inks. Therefore, physically, these particles may perform like tattoo ink particles in the dermis.

FDA regulation. The United States Food and Drug Administration does not currently regulate tattoo procedures and tattoo inks. However, some precautions and guidelines have been published, for example at http://vm.cfsan.fda.gov/-dms/cos-204.html.

4. Optical Imaging Scheme of a Tattoo Dot Under Skin

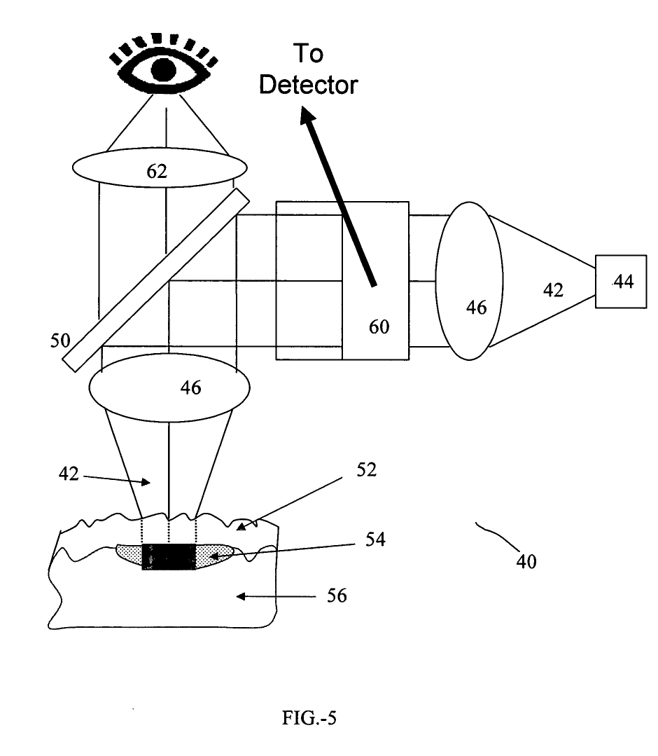

According to one embodiment of the present invention, a tattoo dot serves as a SERS scattering agent. The tattoo dot may have a diameter in the range of approximately 0.1 to 5 mm. The diameter is advantageously approximately 1 mm. In practice, several tattoo dots may be implanted. Typically, only a single dot is probed at a time. However, the present invention also encompasses a method and system for probing multiple dots simultaneously to detect and quantify one or more analytes in vivo. FIG.5 illustrates an apparatus 40 according to one embodiment of the invention, in which Raman scattering from tattoo particles in the top layer of the dermis is measured. The apparatus 40 as shown in FIG.5 may, for example, be mounted in the housing 10 shown in either FIG.1A or FIG.1B. As discussed above, the inner lower and inner upper arm areas are advantageous locations for implantation of silver nano-particles, since these locations are accessible and convenient for locating an external sensor and because the epidermis is generally relatively thin and light-colored (lightly pigmented) at these locations on the body. Referring to FIG.5, a beam 42 from an excitation light source such as a laser 44 passes through a path optionally comprising one or more focusing lenses 46, fiber optic cables (not shown), and a 45° mirror 50 before reaching the epidermis 52. The beam passes through the thin epidermis 52 and reaches the tattoo particles in a tattoo spot 54 in the upper layer of the dermis 56. The surface enhanced Raman radiation emitted by the sensitized particles embedded in the tattoo spot 54 is collected by a focusing lens 46 and directed through a beam splitter 60 to a fiber bundle (not shown) leading to a detector (not shown). The Raman scattered light exits through the epidermis in a wide range of angles, since Raman scattering is an isotropic process. The Raman light is collected by optics to couple the Raman radiation into the fiber bundle. Because the nano-particle tattoo lies under a thin layer of epidermis, absorption and scattering losses in the epidermis are expected to be small. FIG.5 further illustrates an optional feature of the present invention: an eye viewing port 62 for finding the tattoo spot 54 and properly aligning the incident beam 42. The 45° mirror 50 may advantageously have a high reflection coefficient for near-IR radiation, for example from approximately 800 to 1000 nm but near-100% transmission of visible light. Above the 45° mirror 50 is mounted an eyepiece 62 for imaging the tattoo spot. A cross-hair reticule (not shown) may be added to the optics to aid alignment of the instrument to the tattoo spot 54.

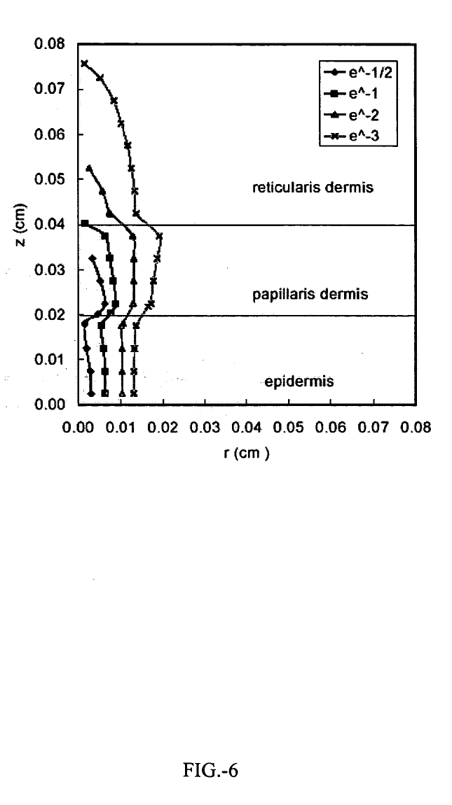

The simulation results and the contour lines of absorbed energy in the z-r symmetrical plane for a published (Zuomin Zhao, “Pulsed Photoacoustic Techniques and Glucose Detection in Human Blood and Tissue” Ph.D. thesis, University Oulu, Finland 2002 p61) skin model are shown in FIG.6 for a narrow beam. The maximal absorption is located on a portion of the z-axis that is in the papillary dermis, not at the surface of the skin. This is mainly because the maximal absorption coefficient of the dermis is just under the epidermis. As shown in FIG.6, a narrow laser beam produces a cylindrical photo-absorption zone located less than a few tenths of a mm from the skin's surface. In this case, the transmission medium is approximately 0.2 mm of the epidermis which causes a very minor change to the light path. In the upper dermis region (papillary dermis), there is a greater degree of scattering loss. However, scattering losses have little impact on the Raman scattering signal, because of the thinness of the scattering layers.

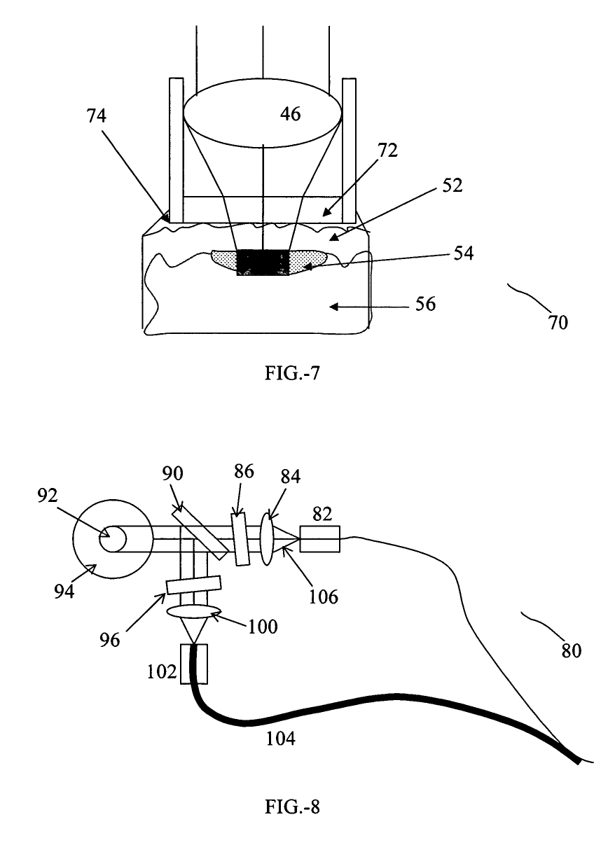

In the simulation of FIG.6, the skin surface was assumed to be smooth. Actually, the roughness of skin may present difficulties due to changing refraction indexes that lead to increased scattering of the incident light beam. A grease or gel with a refraction index matched to the epidermis, approximately n=1.5, may be applied to the skin to smooth out wrinkles and rough surfaces and to eliminate air packets and other sources of refraction index discontinuities. In this manner, the gel reduces the amount of light scattered at the interface between the skin and the environment. An example of an apparatus 70 according to this embodiment of the present invention is shown in FIG.7. A the apparatus of FIG.5 may further comprise a window 72 may be pressed on the skin with matching grease or gel 74 between the window and the upper surface of the epidermis 52. Such pressing of the window to the skin facilitates a smooth transition from the window to the skin without drastic changes in refractive index. The incident beam 42 passes through the window 72 into the skin to the tattoo nano-particles 54 in the dermis 56.

5. Optical Spectral Analysis

The following description presents two possible, exemplary embodiments of the present invention: of the, in one embodiment, an arm monitor head is separated from the laser and spectrograph and in an alternative embodiment, the arm monitor head is co-mounted with the laser and spectral monitor. FIG.8 shows a top view of an apparatus 80 accoridng to one embodiment of the invention with fiber couplings. In general, the skin is illuminated by radiation emitted from a launching fiber 82 which passes through a lens 84, bandpass filter 86, and beam splitter 90 before being reflected by the 45-degree mirror 92 to illuminate the skin. Raman radiation emitted by nano-particles embedded in the dermis of the skin is collected by a lens 94 oriented parallel to the skin (in the plane of the page), is reflected by the 45 degree mirror 92, and is then deflected by the beam splitter 90. The collected Raman radiation then passes through a notch filter 96 and a focusing lens 100 and is collected by a fiber bundle 102. The excitation laser is coupled into the monitor head via a single mode fiber 104. The Raman signal is coupled into a fiber bundle for feeding to a grating based spectrograph. The band pass filter 86 is used to improve the side mode suppression of the probe beam 106 which illuminates the tattoo particles (not shown). Raman generation within the launch fiber 82 and/or side mode emission from the laser can degrade the side mode suppression of the probe beam, and the bandpass filter 86 reduces the impact of these effects on instrument performance. The beam splitter 90 passes excitation laser light, advantageously having a wavelength of about 830 nm, while advantageously reflecting light with wavelengths longer than 866 nm. A super notch filter with a blocking power factor of approximately 10⁶ (i.e., OD 6 at the laser wavelength) may be used to reduce elastically scattered laser radiation entering the fiber bundle. The Raman signal is frequency shifted in a range of approximately 600 to 1800 cm⁻¹, which gives a wavelength range of approximately 873.5 nm to 975.8 nm. The Raman spectrum is advantageously collected using a grating based spectrograph (not shown). The fiber bundle may be used to collect photons for coupling into the spectrograph. Because SERS has a much stronger signal than regular Raman scattering, laser power for the incident excitation beam may be reduced to a lower level and detector requirements may be reduced to a level where lower efficiency, less expensive collectors, such as for example a silicon CCD, may provide sufficient performance. To reduce interference with the measurement from background fluorescence emissions caused by the excitation beam, the laser wavelength may be increased from approximately 830 nm to, for example approximately 980 nm or approximately 1064 nm. Higher wavelength incident radiation produces less fluorescence than lower wavelength (higher energy) light because fluorescence emissions generally scale with the inverse of the fourth power of the incident wavelength. For higher wavelength incident light, an InGaAs detector array may be used in place of the CCD. In a variation from the scheme illustrated in FIG.8, the excitation laser and spectral monitor may be integrated on one board.

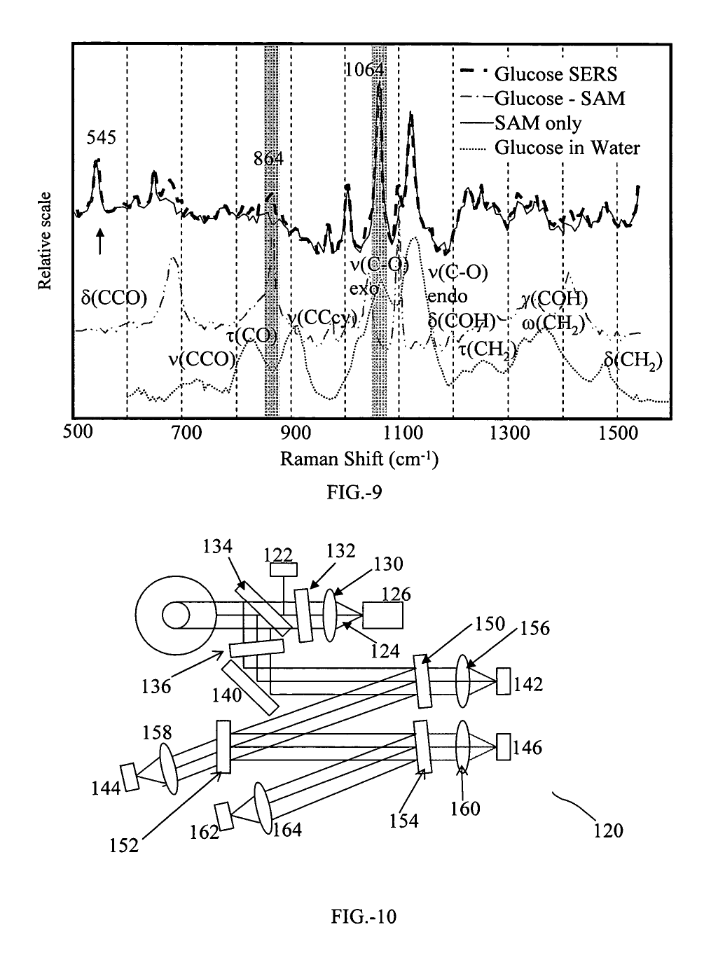

Using SERS, a glucose Raman scattering signal may be selectively enhanced by more than a million times compared to other substances in the tissue. The glucose concentration is human tissue is approximately 100 mg dL⁻¹ while the human tissue total mass is 100 g dL⁻¹. This factor of one million enhancement may elevate the glucose signal to approximately one thousand times that of other tissues, assuming a similar Raman scattering cross section. In fact, the molecules in the SAM are also enhanced as shown in the spectra shown in FIG.9. As shown in FIG.9, some glucose lines are well resolved and separated from SAM, for example those at approximately 1064 cm⁻¹ and approximately 584 cm−1. The thick line shows a SERS Raman spectrum of glucose on a self-assembled monolayer (SAM) substrate attached to silver-coated nano-spheres. The dashed line shows a SERS Raman spectrum of the SAM substrate attached to silver-coated nano-spheres without the presence of glucose (i.e., a background spectrum). The thin solid line shows a convention Raman spectrum of glucose in water. By using a narrow band pass filter, scattering at these wavelengths may be selected for to the exclusion of SAM lines. In addition, a narrow band pass filter may be used to select one of the SAM lines, such as for example approximately 864 cm⁻¹, for use as a standard reference.

In an apparatus 120 according to a further embodiment of the present invention, illustrated in FIG.10, the laser power is monitored as a reference by detector 122. The beam 124 from the excitation light source 126 is provided by diode after a collimating lens 130 and through a band pass filter 132. Some residual light reflected from the beam splitter 134 is monitored as laser power signal. The Raman light after a beam splitter is sent through a notch filter 136. After reflection by an optional angled mirror 140, three bands of Raman spectrum are monitored by three monitors 142, 144, 146 via three respective narrow band pass filters 150, 152, 154 after focusing by three respective lenses 156, 158, 160. Radiation rejected by all three narrow band pass filters 150, 152, 154 is measured as the spectral baseline by a fourth monitor 162 after focusing by lens 164.

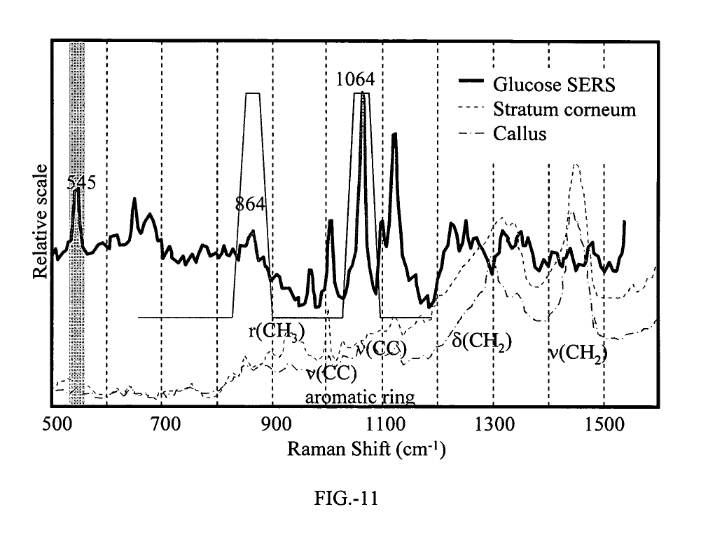

For a laser at 830 nm, the two glucose bands at approximately 545 cm⁻¹ and approximately 1064 cm⁻¹ are at the wavelengths, approximately 869.3 nm and approximately 910.4 nm respectively. The SAM reference line for approximately 864 cm⁻¹ is at approximately 894.1 nm. These three signals are selected by narrow band pass filters A, B, C as shown in FIG.10. The SERS of glucose with SAM on AgFON (thick, solid line) is compared with skin Raman spectra of Stratum corneum and callus (two different dashed lines, as indicated on legend) in FIG.11. Narrow band pass filter functions are plotted against the spectrum of glucose and SAM to indicate one method for isolating peaks. In addition, a detector without a filter is used to monitor the remaining spectrum as background of the spectrum. To avoid fluorescence, the laser wavelength may be increased to a longer wavelength, such as for example approximately 980 nm or approximately 1064 nm.

6. Spectral Lines in Comparison with Background

Glucose spectral lines may be interefered with by scattering from the single molecular layer of SAM on AgFON and possible tissue background. FIG.10 and FIG.11 illustrate glucose SERS analyses compared with AgFON itself, glucose, and skin Raman spectra. In FIG.10, this particular spectrum shows glucose SERS has much stronger signal than SAM does on AgFON. This spectrum was taken at a high glucose concentration of approximately 1800 mg dL⁻¹, which is approximately 1020 times higher than typical physiological levels. One important issue is to find strong glucose lines which are separated from SAM molecular lines since the SAM lines are also strongly enhanced by AgFON plasmon. The 545 and 1064 glucose lines are strong and have little overlap with the SAM lines. In addition, the 864 line from SAM on AgFON has little overlap with glucose lines. FIG.11 also compares the 545, 864, and 1064 lines are with a skin background spectrum, in which the two major components, stratum corneum and callus are shown. The three lines have almost no overlap with any major peaks of the skin background spectrum. FIG.11 also illustrates the band pass filter functions (as shown in the embodiment of FIG.10) covering the three lines.

The spectral signals for two glucose bands (S(A) and S(C)) and one SAM band (S(B)) are first corrected by subtracting off the base line signal S(E) and then normalizing by the laser power S(D). The base line signal is an averaged signal of the spectrum excluding those three bands, which arises mainly from background signal and tissue fluorescence:

| | | | |
|---|---|---|---|
| Glucose signal 545: | G_{I} | = [S(A) − S(E)]/S(D). | (2) |
| Glucose signal 1064: | G_{II} | = [S(C) − S(E)]/S(D). | (3) |
| SAM signal 864: | SAM | = [S(B) − S(E)]/S(D). | (4) |

The Glucose signal is further normalized by the SAM signal, since the SERS glucose signal varies depending on how many silver particles are illuminated by the laser. Glucose signal variation could also come from the variation of how many particles are present in the tattoo spot and/or from the alignment of the laser spot onto the tattoo spot. However, these sources of variation of the glucose signal can all be removed by using the SAM signal (small particle background) as a reference.

| | | | |
|---|---|---|---|
| Glucose concentration 545: | GC_{I} | = G_{I}/SAM | (5) |
| Glucose concentration 1064: | GC_{II} | = G_{II}/SAM | (6) |

The glucose concentration obtained from each line are then averaged with a weighting factor on each one. Because the spectral line intensity and back ground next to the line are different, likely errors of the two bands are different too. As a result, two weighting factors will be derived from spectral analysis and instrumental filter functions. The final glucose concentration signal, GC, is then obtained from:

Glucose concentration:
GC=g′GC_{I}+g″GC_{II} (7)
Where g′ and g″ are weighting factors and the sum of them is 1.

The foregoing description of specific embodiments and examples of the invention have been presented for the purpose of illustration and description, and although the invention has been illustrated by certain of the preceding examples, it is not to be construed as being limited thereby. They are not intended to be exhaustive or to limit the invention to the precise forms disclosed, and obviously many modifications, embodiments, and variations are possible in light of the above teaching. It is intended that the scope of the invention encompass the generic area as herein disclosed, and by the claims appended hereto and their equivalents.

1. A method for in vivo detection of an analyte in a subject, comprising the steps of:
embedding sensitized particles within the skin of the subject, the sensitized particles having an adsorptive affinity for the analyte such that the analyte concentration at the surface of the sensitized particles is greater than the analyte concentration in the subject;
optically illuminating the sensitized particles with excitation radiation;
collecting and measuring optical radiation emitted by the sensitized particles; and
analyzing the emitted radiation to detect the analyte and/or quantify the concentration of the analyte in the subject.

2. The method of claim 1, where the particles comprise nanospheres coated with a silver film.

3. The method of claim 2, where the nanospheres are further coated with a self assembled monolayer which acts to preconcentrate said analyte.

4. The method of claim 1, where the emitted radiation comprises Raman scattered radiation.

5. The method of claim 4, where the Raman scattered radiation comprises surface enhanced Raman scattered radiation.

6. The method of claim 1, wherein the sensitized particles are embedded within the dermis layer of the subject's skin.

7. The method of claim 1, wherein the sensitized particles are embedded in the skin of the subject's inner arm.

8. The method of claim 7, wherein the particles are embedded within the skin of the subject's inner wrist or the skin of the subject's inner upper arm.

9. The method of claim 1, wherein the source radiation is a laser beam.

10. The method of claim 9, further comprising the step of aligning the laser beam to the particles using a means for visual alignment.

11. The method of claim 1, further comprising the steps of:
obtaining an analyte signal A at a first excitation wavelength;
obtaining a small particle background signal BG at a second excitation wavelength;
obtaining a baseline signal BS; and
calculated and correcting an analyte signal substantially equal to (A-BS)/(BG-BS).

12. The method of claim 1, wherein the analyte is glucose.

13. An apparatus for in vivo determination of an analyte, comprising:
a housing, the housing being attachable to a subject's arm by a strap such that a face of the housing is approximately flush with a skin surface on the subject's arm;
an optical path for conveying an incident beam of excitation light from an excitation source and for conveying collected scattered radiation to a detector; the optical path comprising an angled mirror to direct the incident beam approximately perpendicular to the skin, and a focusing lens that focuses the incident beam on a tattoo spot in the skin and focuses the scattered radiation emitted from the tattoo spot.

14. The apparatus of claim 13, wherein the optical path further comprises:
a first optical fiber bundle conveying the incident beam to the housing; the excitation source being external to the housing; and
a second optical fiber bundle conveying the scattered radiation to the detector, the detector being external to the housing.

15. The apparatus of claim 13, further comprising:
an eye viewing port for finding the tattoo spot and for properly aligning the incident beam, the eye viewing port further comprising an eye viewing port, the eye viewing port being mounted in the housing on a side of the housing opposite the face of the housing that lies flush with the skin such that the focusing lens, the angled mirrir, and the viewing port for a linear optical path to the skin.

16. The apparatus of claim 15, further comprising:
a cross-hair reticule for aligning the incident beam with the tattoo spot.

17. The apparatus of claim 15, wherein:
the angled mirror has a high reflection coefficient for near-infrared radiation and a near-100% transmission of visible light.

18. The apparatus of claim 13, wherein:
the incident light beam provides near-infrared excitation light.

19. The apparatus of claim 13, further comprising:
a window positioned in the face of the housing that mounts flush to the skin, the window having a refractive index that is substantially similar to that of the skin, the window providing a surface near the skin onto which a grease may be applied to reduce discontinuity in refractive index between the skin surface and the window.
